(19) United States
(12) Patent Application Publication
(10) Pub. No.: US 2024/0142474 A1
(43) Pub. Date: May 2, 2024

(54) Opsins as Chemosensory Receptors and Related Methods

(71) Applicant: The Regents of the University of California, Oakland, CA (US)

(72) Inventors: Craig Montell, Santa Barbara, CA (US); Yinpeng Zhan, Santa Barbara, CA (US); Avinash Chandel, Santa Barbara, CA (US)

(73) Assignee: The Regents of the University of California, Oakland, CA (US)

(21) Appl. No.: 18/547,840

(22) PCT Filed: Feb. 24, 2022
(86) PCT No.: PCT/US2022/017747

§ 371 Date: Aug. 24, 2023

Related U.S. Application Data
(60) Provisional application No. 63/152,939, filed on Feb. 24, 2021.

(51) Int. Cl. G01N 33/74; A01K 67/0276

(52) U.S. Cl. CPC G01N 33/74; A01K 67/0276; A01K 2217/054; A01K 2227/105; G01N 2333/726

(57) ABSTRACT

Opsin proteins are GPCRs known for light perception. Here it is shown that these receptors are also expressed in chemosensory cells and function as chemosensory receptors activated by a range of molecules, including tastants such as theobromine, for which a receptor has not previously been known. Provided are novel assay systems for assessing chemosensory opsin activation by chemical ligands. By these assays, novel ligands of chemosensory opsins may be identified, the chemosensory properties of molecules may be evaluated; and the modulation of chemosensory properties by modifications or modulators may be tested.

### CROSS-REFERENCE TO RELATED APPLICATIONS

This application claims the benefit of priority to U.S. Provisional Patent Application Ser. No. 63/152,939 entitled “Opsins as Chemosensory Receptors and Related Methods,” filed Feb. 24, 2021, the contents of which are hereby incorporated by reference.

### STATEMENT REGARDING FEDERALLY SPONSORED RESEARCH OR DEVELOPMENT

This invention was made with government support under grant number R01DC016278 awarded by The National Institutes of Health. The government has certain rights in the invention.

### BACKGROUND OF THE INVENTION

G protein-coupled receptors (GPCRs) are membrane proteins that transduce extracellular signals to intracellular responses. Over 700 GPCRs are found in humans. Among their many roles, certain GPCRs are central to the perception of light, taste, and olfaction. For example, the rhodopsin family of GPCRs are light sensitive proteins found in the photoreceptor cells of the retina that facilitate vision and circadian rhythms. Rhodopsins are made up of a GPCR protein subunit called opsin, wherein the opsin is bound to a photosensitive molecule, called 11-cis-retinal (11-cis-retinaldehyde), a pigmented molecule derived from vitamin A. Several different opsins are present in the retina.

Gustatory perception is also facilitated by GPCRs, wherein the GPCRs are present in the taste receptor cells of the taste buds. Type II taste receptor cells express GPCRs which detect sweet, bitter, and umami tastes. Each of each the various GPCR types expressed in taste receptor cells has specificity for a different suite of chemical ligands known as tastants. Binding of a tastant ligand to a compatible GPCR taste receptor results in activation of the GPCR, triggering various cellular responses such as increased cAMP levels, depolarization, potassium and calcium fluxes, and the release of neurotransmitters. Neurotransmitters in turn induce signals that are relayed to the gustatory cortex of the brain.

Thus, gustatory perception of complex chemical signals is achieved by the expression of multiple types of GPCR taste receptors. For example, in humans, there are twenty-five known taste receptors for detection of bitter taste. A large suite of in vitro tools for measuring chemosensory properties of chemical entities is being developed, wherein GPCR taste-sensitive proteins are used to identify and evaluate the gustatory taste attributes of tastant ligands. This field continues to expand, and there is an ongoing need in the art for improved tools to better elucidate the complexities of taste perception.

Olfaction is likewise mediated by GPCRs. In the olfactory epithelium, binding by chemical entities called olfactants results in GPCR activation and generation of signals that enable perception of smell. As with the study of gustatory perception, there is an ongoing need in the art for improved tools to better elucidate the complexities of olfactory perception

Meanwhile, in the food and beverage industry, medical industry, and other areas such as personal care products and scents, the chemosensory properties of products, i.e. flavor and smell, are of utmost importance. The global flavors and fragrances market size is estimated to be at least $25 billion dollars per year. Massive commercial efforts are expended in the development of products having appealing taste and smell (e.g. food products) or tolerable flavor (e.g. medicines). These efforts would be greatly aided by new in vitro tools for screening and characterizing tastants beyond the range of extant assays. Thus, there is a strong and ongoing need in the art for novel screening systems that enable the reliable and efficient characterization of chemosensory properties of various tastants and olfactants.

### SUMMARY OF THE INVENTION

The inventions disclosed herein are based upon the discovery that certain opsin proteins function as chemosensory receptors. Rhodopsins were discovered in the 1870s, and during the nearly 150 years that has elapsed since their discovery, opsin proteins were thought to function exclusively in light perception. As disclosed herein, the inventors of the present disclosure have surprisingly determined that certain opsins, for example, OPN1SW and OPN3, are capable of being activated by non-light stimuli, by the binding of various chemical ligands. Furthermore, the inventors of the present disclosure have demonstrated that expression of these opsins occurs in Type II taste receptor cells in mammalian taste buds, wherein they act as functioning taste receptors. Additionally, it is shown herein that eliminating expression of these opsins in animals results in altered taste reception.

As disclosed herein, the inventors of the present disclosure have further determined that chemosensory opsins such as OPN1SW and OPN3 are activated by a range of tastants, including many important flavonoids, alkaloids and other tastants commonly found in food. As an illustrative example, OPN1SW was found by the inventors of the present disclosure to be activated by theobromine, a bitter alkaloid of the cacao plant which is among the primary compositions giving chocolate its flavor. Previously, no known taste receptor for this chemical was known.

Accordingly, the discoveries disclosed herein provide the art with important tools for chemosensory screening and characterization assays, which may be applied in the development of products having desirable taste and olfactory properties.

In a first aspect, the scope of the invention encompasses GPCR activation detection elements comprising chemosensory opsins such as OPN1SW or OPN3, wherein ligand binding by tastants other chemical entities results in a detectable signal. The scope of the invention further encompasses assay systems for using these detection elements to assess the activation of chemosensory opsins by tastants, olfactants, a complex samples.

In another aspect, the scope of the invention further encompasses various methods of using opsins activation assays. The assays may be employed for various purposes, for example, for the identification of chemical ligands that activate opsins, such as for the purpose of identifying chemicals having desired taste and olfactory properties. In another implementation, the opsin activation assays of the invention are utilized in methods for characterizing the taste or olfactory properties of a composition or material. In another aspect, the scope of the invention encompasses use of chemosensory opsin activation assays to test modifications to or modulators of tastants.

In yet another aspect, the scope of the invention encompasses genetically modified animals wherein chemosensory opsin expression has been modulated. These animal models may be used in behavioral assays known in the art for the improved evaluation of tastants and olfactants.

The foregoing inventions and their various implementations are described in more detail next.

### BRIEF DESCRIPTION OF THE DRAWINGS

FIG. 1A-1G depict TANGO assay results demonstrating activation of OPN3 by different tastant ligands, measured as fold-change from baseline luciferase signal vs. log concentration of tastant. FIG. 1A: asulfame potassium (Ace-k). FIG. 1B: arbutin. FIG. 1C: Phenyl-β-D-glucopyranoside (phe-β-G). FIG. 1D: amygdalin. FIG. 1E: naringin. FIG. 1F: noscapine. FIG. 1G: epicatechin.

FIG. 2A-2F depict TANGO assay results demonstrating activation of OPN1SW by different tastant ligands, measured as fold-change from baseline luciferase signal vs. log concentration of tastant. FIG. 2A: asulfame potassium (Ace-k). FIG. 2B: limonin. FIG. 2C: theophylline (theophy). FIG. 2D: creatinine (creat). FIG. 2E: theobromine. FIG. 2F: genestein.

FIG. 3A-3F depict behavioral taste preference study results wherein mice on a water restricted diet were given access to water with bitter tastants at varying concentrations. Results are expressed as the ratio of tastant licks to pure water licks for wild type mice, measured by lickometer. FIG. 3A: naringin. FIG. 3B: noscapine. FIG. 3C: theobromine. FIG. 3D: limonin. FIG. 3E: theophylline. FIG. 3F: denatonium benzoate.

FIG. 4A-4F depict behavioral study results wherein mice on a water restricted diet were given access to water with bitter tastants at varying concentrations. Results are expressed as the ratio of tastant licks to pure water licks, measured by lickometer, for wild type and OPN3 knockout mice. OPN3¹ and OPN3² are two different OPN3 knockout mouse lines. FIG. 4A: naringin, OPN3¹ mice. FIG. 4B: quinine, OPN3¹ mice. FIG. 4C: denatonium benzoate, OPN3¹ mice. FIG. 4D: naringin, OPN3² mice. FIG. 4E: quinine, OPN3² mice. FIG. 4F: denatonium benzoate, OPN3² mice. n=8, error bars=SEM, * indicates significance at P<0.05, ** indicates significance at P>0.01.

FIG. 5 depicts predictions of potential chemical ligand binding sites in OPN3, based. The retinal docking site is indicated.

FIG. 6A-6E depict TANGO assay results demonstrating activation of OPN3 by different tastant ligands, measured as fold-change from baseline luciferase signal vs. log concentration of tastant. FIG. 6A: quercetin. FIG. 6B: catechin. FIG. 6C: epigallocatechin gallate. FIG. 6D: myricetin. FIG. 6E: asulfame potassium (Ace-k).

FIG. 7A-7C depict behavioral study results wherein mice on a water restricted diet were given access to water with bitter tastants at varying concentrations. Results are expressed as the ratio of tastant licks to pure water licks for wild type and OPN3 knockout mice, measured by lickometer. 7A: Catechin. 7B: epigallocatechin gallate. 7C: Quercetin. n=6, error bars=SEM, * indicates significance at P<0.05, ** indicates significance at P>0.01.

FIG. 8 depicts behavioral study results wherein mice on a water restricted diet were given access to water with narangin at varying concentrations. Results are expressed as the ratio of narangin licks to pure water licks for wild type mice and TRPM5 knockout mice, measured by lickometer. n=6, error bars=SEM, * indicates significance at P<0.05, ** indicates significance at P>0.01.

### DETAILED DESCRIPTION OF THE INVENTION

### I. CHEMOSENSORY OPSIN ACTIVATION DETECTION ELEMENTS AND ASSAYS

The scope of the invention encompasses novel GPCR activation assays wherein the activation of chemosensory opsins by chemical species may be detected. The assays of the invention provide the art with various tools for identifying and evaluating the chemosensory properties of tastants, olfactants (also called odorants), and other molecules. The basic unit of these assays is what will be referred to herein as a “chemosensory opsin activation detection element.” At the most general level, a chemosensory opsin activation detection element comprises a chemosensory opsin and is configured such that activation of the chemosensory opsin by a chemical species results in the generation of a detectable signal. The detectable signal may be any measurable signal, for example, an optical or electrical response to activation of the opsin, for example, mediated by chemical, electrical, or biological activities. These fundamental detection units are used in assay systems to enable the various applications of the invention described herein.

Chemosensory Opsins. The chemosensory opsin activation detection elements of the invention comprise one or more types of chemosensory opsin. A chemosensory opsin is any opsin protein which is activatable by the binding of a ligand comprising a chemical species. The chemical species may comprise any molecule, for example, a tastant or olfactant. In one embodiment, the chemosensory opsin is OPN1SW. In one embodiment, the chemosensory opsin is OPN3. In alternative implementations, the chemosensory opsin may be OPN4 or OPN5.

In a primary implementation, the opsin utilized in the assays and methods of the invention is a human opsin protein. Accordingly, the opsins referred to herein will be called by their human sequence identifiers, e.g. OPN1SW or OPN3. However, it will be understood that reference to a particular opsin also encompasses all homologs, paralogs, and orthologs of the enumerated protein that are found in other species, for example, opsin counterparts of the enumerated protein found in other mammals such as mice, rats, cats, dogs, pigs, horses, guinea pigs, non-human primates, or other animal species.

In some implementations, the selected opsin, e.g., OPN1SW or OPN3, comprises a native opsin protein sequence. It will be understood that references to a particular opsin made herein will also encompass variants of the enumerated protein. A “variant,” as used herein, encompasses modified forms of the enumerated protein, including: active fragments thereof; truncated sequences; sequences comprising amino acid substitutions, insertions, and deletions; fusions with other elements; and other variations or derivatives of the native amino acid sequence, wherein such variant retains one or more ligand binding activities of the native sequence. In some implementations, the variant comprises a ligand binding subsequence or ligand binding domain of the native sequence. In one embodiment, the variant comprises an amino acid sequence having at least 90%, at least 95%, or at least 99% sequence identity to the native sequence of the selected opsin.

In one implementation, the chemosensory opsin utilized in the compositions and methods of the invention comprises OPN1SW, also known as blue-sensitive opsin 1 or short wavelength sensitive opsin 1. In one embodiment, the OPN1SW is human OPN1SW, Uniprot Accession Number P03999. In other embodiments, the opsin comprises OPN1SW from a non-human species, for example OPN1SW homologs from chimpanzee, Rhesus monkey, dog, cow, mouse, rat, or zebrafish. In other embodiments, the chemosensory opsin comprises a variant of OPN1SW or a variant of an OPN1SW counterpart in other species. In one embodiment, the chemosensory opsin comprises an amino acid sequence having at least 90%, at least 95%, or at least 99% sequence identity to OPN1SW.

In one implementation, the chemosensory opsin utilized in the compositions and methods of the invention comprises OPN3, also known as encephalopsin or panopsin. In one embodiment, the OPN3 is human OPN3, Uniprot Accession Number Q9H1Y3. In other embodiments, the opsin comprises OPN3 from a non-human species, for example OPN3 homologs from chimpanzee, Rhesus monkey, dog, cow, mouse, rat, chicken or zebrafish. In other embodiments, the chemosensory opsin comprises a variants of OPN3 or a variant of an OPN3 counterpart in other species. In one embodiment, the chemosensory opsin comprises an amino acid sequence having at least 90%, at least 95%, or at least 99% sequence identity to OPN3.

The selected opsin will typically be present in large numbers per detection element, for example, thousands, tens of thousands, hundreds of thousands, or millions of copies per cell or other detection element. In a primary implementation, each detection element comprises a single type of chemosensory opsin, e.g. OPN1SW or OPN3. In an alternative implementation, two or more different types of chemosensory opsins are expressed or otherwise present in the detection element. In one such implementation, the cell or other detection element may be configured such that each type of chemosensory opsin present generates a distinguishable signal, enabling multichannel detection of activating ligands for different opsins in a single detection unit.

Cell-Based Chemosensory Opsin Activation Detection Elements. In a primary implementation, the chemosensory opsin activation detection element is a cell expressing one or more chemosensory opsins, for example, OPN1SW or OPN3. The cells may be of any species and type.

In one implementation, the cells are chemosensory cells derived from an animal. For example, the cells may comprise explanted sensory tissues or cultured cells derived therefrom. For example, cells may comprise primary taste receptor cells derived from explanted papillae tissue; cultured taste receptor cells; immortalized taste receptor cells; primary olfactory cells derived from explanted olfactory epithelium; cultured olfactory receptor cells; immortalized olfactory cells; and other cells comprising or derived from chemosensory tissues of an animal.

In another implementation, the cell is an engineered cell, wherein the cell is engineered to express one more chemosensory opsins, for example, OPN1SW or OPN3. For example, the cell engineered to express a selected chemosensory opsin may be a human cell, a cell of another mammalian species, a yeast cell, an insect cell, or E. coli or other bacterial cell types. Exemplary cell lines which may be transformed to express chemosensory opsins include HEK293 cells, Expi293 cells, CHO cells, ES0 cells, EB66 cells, Per.C6 cells, and other cells amenable to cell culture, as known in the art.

In an alternative embodiment, the transformed cells of the invention comprise chemosensory cells wherein the expression of a selected native chemosensory opsin, e.g. OPN1SW or OPN3, is modulated, i.e. overexpressed or inhibited. In one embodiment, the cell comprises inhibited expression of a native chemosensory opsin, e.g. OPN1SW or OPN3, for example wherein gene knockout or knockdown has been achieved by methods such as CRISPR/Cas9 systems, short interfering RNA (siRNA), short hairpin (shRNA), and other methods known in the art.

Non-Cellular Chemosensory Opsin Activation Detection Elements. It will be understood that the detection elements of the invention are not limited to cells. In some implementations, the chemosensory opsin activation detection element comprises a non-cellular composition of matter wherein one or more chemosensory opsins, e.g. OPN1SW or OPN3, is present and wherein activation of the opsins results in the generation of a detectable signal. Exemplary non-cellular platforms for GPCR activation assays include, for example: isolated cell membranes; artificial cells; membrane mimetics; detergent micelles; liposomes; and other non-cellular compositions of matter comprising a membrane or membrane-like structure wherein GPCR activation may be achieved and detected.

Detectable Signals. The chemosensory opsin activation detection elements of the invention are configured such that activation of chemosensory opsin proteins therein, e.g. OPN1SW or OPN3, by a chemical species is detectable. The detection of activation may be measured by any effect resulting from GPCR activation by a chemical ligand. For example, the detection element may be configured for generation of a detectable signal resulting from G-protein activity, second messenger generation, electrical or ionic responses, release of neurotransmitters, or any other downstream effect of GPCR activation. The detectable signal may be, for example, any of an optical signal (e.g. light emission or changes in fluorescence) an electrical signals, or a chemical signal.

Calcium. In one embodiment, chemosensory opsin GPCR activation is detected by measurements of calcium status or flux, for example wherein activation of the chemosensory opsin triggers a measurable release or influx of calcium. In such systems, the cells or other detection elements may be pre-loaded with a calcium indicator, such as a calcium-sensitive dye, wherein the fluorescence or emission spectra of the dye is determined by intracellular calcium levels. Exemplary calcium sensing dyes include, for example, Fluo-4, Fluo-4-M, Fluo-8, BCECF, BCECF-AM, Fura-2, Fura-2-AM, calcium indicator calcium green 1 dextran, calcium orange, and other calcium indicators known in the art. In some embodiments, the detection is achieved by a ratiometric fluorescence assay, as known in the art. For example, the excitation spectra of calcium indicator Fura-2 shifts from a wavelength of ˜380 to ˜340 upon calcium binding, and the ratio of the emissions at these two wavelengths enables measurement of the intracellular Ca² concentration. In some embodiments, the calcium indicator is a single wavelength indicator, wherein fluorescence intensity of the indicator is proportional to the intracellular Ca²⁺ concentration. In another implementation, the calcium indicator is a genetically encoded calcium indicator (GECI), as known in the art. Exemplary GECIs include aequorin and fluorescent proteins (e.g. GFP) fused to calmodulin or other calcium binding elements, wherein calcium binding alters the conformation and fluorescence of the protein.

Accordingly, in one embodiment, the chemosensory opsin activation detection element comprises a cell comprising a chemosensory opsin and a calcium indicator, i.e. a chemical species wherein its properties are responsive to calcium concentration or a reporter activated or modulated in response to calcium concentration. In one embodiment, the detection element comprises a cell expressing OPN1SW or OPN3, wherein the cell is loaded with or engineered to express a calcium indicator, for example, a calcium-sensitive dye or GECI. Calcium indicator loading, e.g. dye loading, may be achieved by methods known in the art at concentrations suitable for detection by a selected detection means.

cAMP. In another implementation, the chemosensory opsin activation detector is configured such that activation of the chemosensory opsin is measurable by its effects on cAMP intracellular concentration, for example, inducing a decrease in cAMP. Changes in cAMP concentration may be detected by a cAMP indicator, i.e. a chemical species wherein its properties are responsive to cAMP concentration or a reporter activated or modulated in response to cAMP concentration. Exemplary cAMP indicators for measurement of cAMP dynamics include, for example, dyes such as Dibutyryl-cAMP or genetically encoded cAMP sensors, such as FlCRhR and FRET systems, known in the art. Additionally, cAMP indicator may comprise a composition that responds to changes in intracellular ATP, which is modulated by cAMP. For example, in one embodiment the cAMP indicator is luciferase, which generates light according to intracellular concentrations of ATP.

Accordingly, in one embodiment, the chemosensory opsin activation detector comprises a cell comprising a chemosensory opsin and a cAMP indicator, for example, a cell expressing OPN1SW or OPN3 wherein the cell is loaded with or engineered to express a cAMP indicator.

Beta-Arrestin Recruitment. In one implementation, the chemosensory opsin activation detection element comprises a cell configured such that beta-arrestin recruitment to activated opsin generates a detectable signal, Various GPCR activation assays utilizing beta-arrestin recruitment to generate a signal are known in the art. For example, in one implementation, the detection element comprises an engineered system comprising a first interaction partner fused to the opsin GPCR and a second interacting partner fused to beta-arrestin. Activation of the opsin results in recruitment of beta-arrestin to the opsin and induces interaction between the first and second interacting partners to generate a detectable signal. For example, the signal may be generated by complementary reporter molecule fragments such as complementary beta-galactosidase fragments. In another embodiment, the system is engineered to generate a bioluminescence resonance energy transfer (BRET) signal, wherein a first chromophore is fused to the C-terminus of the chemosensory opsin and a second chromophore is fused to beta-arrestin, wherein ligand binding induces beta-arrestin recruitment to the opsin, in turn creating a measurable signal from the interaction of the two chromophores, for example, measurable by fluorescence microscopy.

For example, in one implementation the chemosensory opsin activation detection element comprises a TANGO assay (Transcriptional Activation Following Arrestin Translocation), as known in the art, for example, as described by Kroeze et al., 2015. PRESTO-Tango as an open-source resource for interrogation the druggable human GPCRome. Nat Struct Mol Biol 22, 362-369. In the TANGO systems of the invention, a cell is engineered to express a chemosensory opsin in the form of a fusion protein, wherein the opsin fusion protein comprises an optional membrane trafficking signal (e.g. HA), an optional FLAG tag or like epitope to facilitate detection, and a transcriptional activator which induces the expression of a reporter gene, such as luciferase, wherein the transcriptional activator is joined to an intracellular portion of the opsin, for example, the N-terminus, by a cleavage sequence. The cells of the TANGO system are further engineered to express a second fusion protein comprising a beta-arrestin fused to a protease, for example, a TEV protease, wherein the protease is capable of cleaving the fusion protein at the cleavage site. The cells of the TANGO system are further engineered to express a reporter protein, e.g. luciferase, under the control of a transcriptional activator, for example, tetracycline-controlled transactivaor (tTA). Ligand binding to the opsin deployed in the TANGO system triggers a conformational change in the opsin that recruits the beta arrestin-protease fusion protein to the opsin, where proximity of the protease induces cleavage of the transcriptional activator, which trafficks to the nucleus and induces the expression of the reporter gene. The TANGO system advantageously allows direct quantification of opsin activation, independent of g protein activity.

Electrical Signals. In one implementation, the chemosensory opsin activation detection element is configured such that activation of the chemosensory opsin is measurable by its effects on electrical properties of cells. For example, voltage sensitive dyes or genetically encoded voltage sensors (GEVIs), or other indicators of electrical activity may be used to detect and measure opsin-activation mediates changes in action potential, cell polarization or depolarization, or other cellular electrical responses induced by GPCR activation. Exemplary voltage-sensitive dyes include aminonaphthylethenylpyridinium (ANEP) and analogs thereof, and tetramethylrhodamine methyl ester (TMRM). Alternatively, changes in electrical status induced by opsin activation may be monitored by patch-clamp recordings, field effect transistors, electrodes, or like devices and methods.

Accordingly, in one embodiment, the chemosensory opsin activation detection element comprises a cell comprising a chemosensory opsin and an indicator of electrical activity, for example, a cell expressing OPN1SW or OPN3, wherein the cell is loaded with or engineered to express an indicator of electrical activity.

Neurotransmitter release. In one implementation, the chemosensory opsin activation detector is configured such that activation of the chemosensory opsin is measurable by its induction of neurotransmitter release. For example, activation of the chemosensory opsin in the cells may result in the measurable release of neurotransmitters such as 5HT, GABA, and acetylcholine. Exemplary systems for detecting neurotransmitter release are described, for example, in Romanov, et al., Afferent neurotransmission mediated by hemichannels in mammalian taste cells. EMBO J. 2007, 26, 657-667 and Dando et al., Acetykholine is released from taste cells, enhancing taste signalling. J. Physiol. 2012, 590, 3009-3017.

Exemplary Embodiments. In one implementation, the scope of the invention encompasses a chemosensory opsin detection element comprising a selected chemosensory opsin, wherein the detection element is configured to generate a detectable signal upon activation of the opsin. In one embodiment, the chemosensory opsin is OPN1SW, an OPN1SW homolog, paralog, or ortholog, or a variant of OPN1SW. In one embodiment, the chemosensory opsin is OPN3, an OPN3 homolog, paralog, or ortholog, or a variant of OPN3. In one embodiment, the detection element comprises a cell. In one embodiment, the cells is a explanted native chemosensory receptor cell, cultured chemosensory receptor cell, or cell culture product thereof, for example, a spheroid or organoid. In one embodiment, the cell is a cell engineered to express the selected chemosensory opsin. In one embodiment, the detection element comprises a non-cellular body. In one embodiment, the detection element is loaded with or expresses a calcium indicator. In one embodiment, the detection element is loaded with or expresses a cAMP indicator. In one embodiment, the detection element is loaded with or expresses an electrical indicator. In one embodiment, the detection element is loaded with or expresses reporter of neurotransmitter activity. In one embodiment, the detection element generates a detectable signal by beta-arrestin recruitment to activated chemosensory opsin.

Assay Systems. The scope of the invention encompasses assay systems and methods of use thereof wherein the foregoing chemosensory opsin activation detection elements are utilized to detect ligand activation of chemosensory opsins. An assay system, in the context of the invention, is a system, i.e. a combination of elements or components, that are utilized in combination to present samples to chemosensory opsin activation detection elements and measure activation signals generated thereby. The general elements of an assay system are:
a containment element comprising a vessel or other structural element for supporting or containing a plurality of chemosensory opsin activation detection elements and configured to receive a sample comprising chemical species;
a plurality of chemosensory opsin activation detection elements disposed within the containment element; and
a detection device for measuring signals generated by activation of chemosensory opsin activation detection elements.

The containment element may comprise any number of configurations. In a primary implementation, the containment element comprises a well, for example, the well of a multiwell plate. In one implementation, the assay systems of the invention may advantageously be scaled massively parallel high throughput screening of hundreds or thousands of separate samples. In one implementation, the containment element comprises microfluidic elements for introducing samples to the chemosensory opsin activation detection elements contained therein.

The assays systems will further comprise suitable detection device or devices for measuring the signals generated by the selected chemosensory opsin activation detection elements. Optical signals may be detected by devices such as photodetectors, fluorescent microscopy systems, and optical plate readers as known in the art. Electrical signals may be detected by voltammetry, amperometric, potentiometric, or piezoelectric sensors such as electrodes or field effect transistors.

Assay systems of the invention may further encompass data collection, storage, and analysis elements for processing signals generated by ligand binding to chemosensory opsins.

Exemplary GPCR Activation Assay Systems. Various GPCR activation assays are known in the art and may be reconfigured or adapted to detect activation of a chemosensory opsin, e.g. OPN1SW or OPN3, by a chemical species.

In one implementation, the chemosensory opsin activation assay system of the invention comprises detection elements and systems configured to detect GPCR activation by tastants, wherein a chemosensory opsin, e.g. OPN1SW or OPN3 is used as the activatable GPCR. Exemplary systems include, for example, those described in: U.S. Pat. No. 8,004,152, Bioelectronic Tongue, by Fung et al.; United States Patent Application Publication Number 20150037909, Electronic Tongue or Nose Sensors, by Livache et al.; PCI Patent Application Publication Number WO2017212377, Device System and Relating Method for the Quantitative Assessment of Taste Sensitivity, by Barbarossa et al.; Liu et al., Cell-based biosensors and their application in biomedicine. Chem. Rev. 2014, 114: 6423-6461; Podrazka et al., Electronic Tongue-A Tool for All Tastes? Biosensors 2017, 8: 3; Ha, et al., Recent achievements in electronic tongue and bioelectronic tongue as taste sensors. Sens. Actuators B Chem. 2015, 207: 1136-1146; Wu et al., Bioanalytical and chemical sensors using living taste, olfactory, and neural cells and tissues: A short review. Analyst 2015, 140: 7048-7061; Wu, et al., Biomimetic Sensors for the Senses: Towards Better Understanding of baste and Odor Sensation. Sensors 2017, 17: 2881; Wasilewski et al., Bioelectronic tongue: Current status and perspectives. Biosens. Bioelectron. 2020, 150:111923; Woertz, et al., Taste sensing systems (electronic tongues) jar pharmaceutical applications. Int. Pharm. 2011, 417: 256-271; and Gutiérrez, et al., Hybrid electronic tongue based on optical and electrochemical microsensors for quality control of wine. Analyst 2010, 135: 1718-1725.

In another implementation, the chemosensory opsin activation assay system of the invention comprises detection elements and systems configured to detect GPCR activation by olfactants, wherein a chemosensory opsin, e.g. OPN1SW or OPN3 is used as the activatable GPCR. GPCR activation assays known in the art for olfactory evaluation may be utilized, as described in, for example: United States Patent Application Publication Number 20150005177, Methods of identifying, isolating and using odorant and aroma receptors, by Pfister et al.; U.S. Pat. No. 8,377,706, Olfactory receptor-functionalized transistors for highly selective bioelectronic nose and biosensor using the same, by Hong et al.; U.S. Pat. No. 7,291,485, Gaq protein variants and their use in the analysis and discovery of agonists and antagonists of chemosensory receptors, by Yao et al.

### II. METHODS OF THE INVENTION

The scope of the invention encompasses methods of using of the foregoing novel detection elements and assays. The methods of the invention may be applied in numerous contexts and for various purposes, as set forth below, including: measuring chemosensory opsin activation by chemical species; identifying novel ligands of chemosensory opsins; characterizing the chemosensory attributes of tastants, olfactants and other chemical species; and testing the effects of ligand modifications or chemosensory modulators on chemosensory opsin activation.

In a general implementation, the scope of the invention encompasses: a method of measuring activation of a chemosensory opsin by a chemical species, comprising the steps of:
providing a sample to a plurality of chemosensory opsin activation detection elements,
wherein the chemosensory opsin activation detection elements comprises a cell or other composition of matter comprising a selected chemosensory opsin, configured such that activation of the chemosensory opsin generates a detectable signal;
wherein the sample comprises or putatively comprises an activating ligand of the chemosensory opsin;
wherein sample is provided to the chemosensory opsin activation one or more detection elements under conditions suitable for ligand binding to, or interaction with, the chemosensory opsin proteins present therein; and
measuring signal generated by the plurality of chemosensory activation detection elements.

Typically, a plurality of chemosensory opsin activation detection elements are used, e.g. at least a minimal number of detection elements for generation of a sufficient signal to be measurable by the detection device of the associated assay system. In some very high resolution systems, for example, single cell detection systems, only one or a few detection elements may be present. In a typical implementation, the chemosensory opsin activation detectors will comprise cells, and the cells will be present in large numbers, for example, hundreds, thousands, tens of thousands, hundreds of thousands, or millions of cells. The cells may be provided in any number of formats, including, for example: explanted chemosensory tissues derived from an animal; cultured chemosensory cells (e.g. cultured primary taste receptor cells or taste bud cells, or cultured immortalized taste cells); suspensions of cultured cells; cultured cell monolayers; three-dimensional cell culture products such as spheroids and organoids; and other collections of cells in sufficient numbers to generate a measurable signal in response to activation.

In one embodiment, the concentration of chemosensory opsin-activating species in the sample is unknown. In other implementations, the concentration is known. The concentration of chemosensory opsin-activating species in the sample and may be presented to the opsin activation detector in a range of concentrations, for example, at concentrations of 1.0 nM to 10 mM

Exemplary embodiments. In one embodiment, the chemosensory opsin of the detection element comprises OPN1SW or a variant thereof. In one embodiment, the chemosensory opsin of the detection element comprises OPN3 or a variant thereof. In one embodiment, the chemosensory opsin detection element comprises a cell expressing OPN1SW, OPN3, or a variant thereof. In one implementation, multiple types of chemosensory opsin activation detectors (i.e. more than one type of chemosensory opsin) is present in the detection element.

In one embodiment, the sample comprises a tastant or putative tastant. In one embodiment, the sample comprises an olfactant or putative olfactant. In one embodiment, the sample is a substantially homogeneous sample. In one embodiment, the sample is a heterogeneous sample. in one embodiment, the sample is complex sample, for example, comprising food material, beverage, medical formulation, personal care product, or other material. In one embodiment, the sample comprises an activating chemical species in combination with a chemosensory modulator or putative chemosensory modulator. In one embodiment, the sample comprises modified form of an activating species.

In one implementation, the measurement provides a qualitative measurement, wherein, if a detectable signal is generated, the sample is determined to comprise one or chemical species that activates the selected chemosensory opsin of the chemosensory opsin activation detection element. In another implementation, the measurement provides a quantitative measurement, wherein, if a detectable signal is generated, the magnitude of the signal indicates the abundance and/or activation capacity of activating species present in the sample. In one embodiment, a single measurement is obtained per sample. In one embodiment, multiple measurements are obtained over time. In one embodiment, the measurement is a continuous measurement, for example, for monitoring a sample comprising a batch or stream of material, for example in a food processing or manufacturing context.

Identification of Opsin Ligands. The foregoing general assay method may be implemented in various contexts. In a first aspect, the method of the invention is applied for the identification of activators of a chemosensory opsin. For example, a chemical entity, such as a known or novel tastant or olfactant, may be used in an opsin activation assay, wherein it is not known a priori if the chemical entity is an activator of the selected opsin. If activation is observed, the chemical entity is deemed to be an activating ligand of the selected opsin. By this method, ligands of chemosensory opsins may be newly identified. As the discovery of opsins as taste receptors has not previously been explored, this method will enable comprehensive profiling of ligand interactors for chemosensory opsins including OPN1SW and OPN3.

In a related implementation, modeling and in silico approaches may be used to identify putative activators of chemosensory opsins. Based on the known structures of a chemosensory opsin, e.g. OPN1SW or OPN3, and/or the structures of previously identified ligands thereof, in silico modeling tools may be used to identify candidate activators a chemosensory opsin. For example, machine learning or other computational tools for predicting ligand binding interactions may be utilized, such as P2Rank, PRankweb, COACH, and other modeling tools known in the art. In one implementation, the method encompasses: selecting a set of ligands of a selected opsin, wherein the ligands have one or more known taste, smell, or other chemosensory properties; comparing the structural and chemical properties of the ligands by a computerized analysis to determine one or more common structural motifs that facilitate ligand binding to the selected opsin; and wherein a composition of unknown chemosensory properties will be predicted to have chemosensory properties of the known ligands of the selected opsin if the one or more identified structural motifs is present in the composition.

The chemical candidates identified in silico may then tested and verified using in vitro and behavioral assays disclosed herein or other techniques such as evaluation by human tasters administering the chemical to a human tester and determining if the chemical has the putative taste, smell, or other chemosensory property associated with ligands of the selected opsin.

Profiling taste and olfactory properties of a chemical entity. In another implementation, the assay method of the invention may be utilized to characterize the taste and/or olfactory properties of a chemical entity. In one implementation, the method is as follows:
a chemical composition is presented to an activation assay comprising a selected chemosensory opsin, wherein the chemosensory attributes (e.g. taste, smell) of one or more ligands of the selected opsin are known;
the ability of the chemical composition to activate the selected chemosensory opsin is evaluated by means of the activation assay; and
if the chemical composition is to be an activator of the selected chemosensory opsin, the chemical composition is determined to have one or more chemosensory attributes of the known ligand(s) of the opsin.

For example, the inventors of the present disclosure have determined that OPN1SW is activated by theobromine, theophylline, creatinine, limonin, and genistein. It is expected that other ligands of OPN1SW will share one or more chemosensory attributes of these identified ligands. Accordingly, chemosensory attributes of OPN1SW ligands encompass one or more chemosensory attributes selected from: a flavor or olfactory attribute of theobromine, including bitterness (for example, cocoa-type bitterness, tea-type bitterness, and kola nut-type bitterness); a flavor and olfactory attribute of theophylline, including bitterness (for example, cocoa-type bitterness, tea-type bitterness, and kola nut-type bitterness); a flavor and olfactory attributes of creatinine, including bitterness; a flavor and olfactory attributes of limonin, including bitterness (for example, citrus-type bitterness; citrus flavor and scent, for example, orange flavor and scent; pine flavors and scents); and flavor and olfactory attributes of genistein, including bitterness (for example, soy and legume-type bitterness; soy and legume flavor and scent).

The inventors of the present disclosure have determined that OPN3 is activated by arbutin, amygdalin, phe-β-Glu, naringin, noscapine, epicatechin, catechin, Epigallocatechin, and quercetin. It is expected that other ligands of OPN3 will share one or more chemosensory attributes of these identified ligands. Accordingly, chemosensory attributes of OPN3 ligands encompass: flavor and olfactory attributes of arbutin, including bitterness; flavor and olfactory attributes of amygdalin, including bitterness (for example bitter almond type flavor and scent); flavor and olfactory attributes of phe-β-Glu; flavor and olfactory attributes of noscapine, including bitterness (for example, poppy-like bitterness); flavor and olfactory attributes of epicatechin, including bitterness (for example, green tea-like bitterness); flavor and olfactory attributes of catechin, including bitterness (for example, green tea-like bitterness); flavor and olfactory attributes of epigallocatechin, including bitterness; and flavor and olfactory attributes of quercetin.

In some embodiments of the characterization assay, the sample may comprise a putative food additive (e.g. flavorant, texturizer, preservative, bulking agent, or other food ingredient), an orally-administered drug or dietary supplement(e.g. a pharmaceutical composition, nutritional supplement, or other active agent), or an oral care composition (toothpaste, mouthwash, whitening agent, etc.). In other implementations, the olfactory properties of a chemical entity are of interest. The methods of the invention allow in vitro and high throughput screening of chemical entities that activate or modulate chemosensory opsins. For example, as many OPN1SW and OPN3 ligands have a bitter taste, the method may utilized to identify samples that have an objectionable taste and which have desirable or undesirable taste and/or olfactory properties.

In another context, the assays of the invention further provide a monitoring tool, for example, a quality control tool, wherein samples may introduced to the assay to determine if activators of the selected chemosensory opsin of the assay are present and/or to measure the abundance of such activators. For example, samples may be derived from food or beverage processing production streams to ensure bitter flavors or other objectionable chemosensory attributes are not arising.

Modulation of Chemosensory Attributes. In various contexts, it may be desirable to modify the chemosensory attributes (e.g. flavor or scent) of a molecule. In some implementations, it may be sought to enhance a desirable flavor or scent. In a common application, it is sought to mask or eliminate an undesirable flavor or scent. For example, it is often desirable to mask or eliminate bitter flavors found in food products, orally-delivered drugs, and other ingested items. Various ligands of chemosensory opsins, for example activators of OPN1SW and OPN3, comprise molecules with bitter or otherwise objectionable chemosensory attributes. For example, naringin (e.g. present in grapefruit) is a potent activator of OPN3 and elicits a strong bitter taste, wherein removal or masking of this bitterness is desirable in commercial production of grapefruit juice and other products containing naringin.

In product development, modifications of a composition may be applied to alter the chemosensory properties thereof. For example, tastants such as food products or drugs may be modified by treatments intended to mask or eliminate undesirable flavors, such as bitter flavors. Exemplary modifications of tastants include enzymatic treatments (e.g. application of naringinase), microencapsulation in polymeric materials (e.g. wax, zein, gelatin, ethylcellulose, etc.), and chemical complexion with masking agents (e.g. cyclodextrans).

The chemosensory opsin activation assays of the invention provide a means of evaluating such modifications made to tastants, olfactants, and other molecules comprising known activators of a chemosensory opsin, or modifications made to materials containing these activating ligands, in order alter the chemosensory properties thereof. In this implementation, a sample comprising a modified form of a chemosensory opsin activating ligand, (or a modified form of a material containing the activating ligand) is provided to the assay and its capacity to activate a selected chemosensory opsin is measured. The activation capacity is compared to that of the unmodified form of the activating ligand (or material containing the activating ligand), wherein, if the activation capacity is changed by the modification, the modification is deemed to alter one or more chemosensory attributes of the activating species. For example, If the modification reduces the activation capacity of the ligand, the modification is deemed to successfully mask or eliminate chemosensory attributes of the unmodified opsin activating ligand, for example, to the mask bitterness associated with activators of OPN1SW or OPN3. Accordingly, in one embodiment, the method of the invention is utilized to measure the activation of a selected opsin by a sample, wherein the sample is comprises a modified variant of a reference sample having known activation capacity of the selected opsin. By comparing the activation capacity of the variant sample to that of the reference sample, the effect of the modification can be evaluated.

Likewise, in the development of food, medicine, and other products, chemosensory modulators are utilized to enhance positive attributes or mask or eliminate undesirable attributes. A chemosensory modulator, as used herein, comprises a composition of matter that alters the chemosensory attributes (e.g. flavor or scent) of a chemosensory opsin activator. The modulator may, in various implementations, be a modulator that masks or eliminates an undesirable flavor or scent; enhances a desirable flavor or scent, or otherwise alters the flavor, scent, or other chemosensory attribute of a molecule. For example, many activators of OPN1SW and OPN3 are compositions having a bitter taste and it would be desirable to find compositions of matter that may be combined with the activator to mask or eliminate the undesirable taste. Exemplary taste modulators include sweeteners, acidifiers, carbohydrates, lipids, fiber, minerals, and other additives known in the art to modulate the taste properties of a selected tastant.

The methods of the invention provide a means of identifying and evaluating chemosensory modulators. In this implementation, an activation assay of the invention comprising a selected chemosensory opsin, e.g. OPN1SW or OPN3, is provided with a sample comprising a known activating ligand of the selected opsin. The sample further comprises one or more putative chemosensory modulators. The activation capacity of the sample is assessed by means of the assay and the measured activation is compared to that achieved by like samples comprising the activating ligand in the absence of the putative modulator. If the activation capacity of the activator is altered by the addition of the putative modulator, the putative modulator is deemed to modulate one or more chemosensory attributes of the opsin activating composition. For example, if the sample contains a known activator that is a bitter composition, and if addition of the putative modulator to the sample reduces chemosensory opsin activation thereby, the putative modulator is determined to attenuate bitterness of the composition. By application of this general method, novel modulators may be discovered, for example in high throughput screens, or the relative modulating capacity of different chemosensory modulators may be compared. Accordingly, in the method of the invention, in one embodiment, the method is used to assess activation of a selected opsin by a sample comprising the combination of a known activator of the selected opsin and a putative chemosensory modulator comprises a composition that alters the chemosensory attributes of a chemosensory molecule (e.g. a tastant or olfacant). For example, in one embodiment, the known activator of the selected opsin is a bitter composition and the putative modulator is a composition that attenuates bitter flavor, for example, a composition that masks, inhibits, or eliminates bitter flavor

### III. ENGINEERED ANIMALS HAVING MODULATED CHEMOSENSORY OPSIN EXPRESSION

In another implementation, the scope of the invention encompasses an engineered animal, wherein expression of one or more chemosensory opsins is modulated in the animal, relative to that wild type animals.

In a first embodiment the engineered animal is an animal wherein expression of native chemosensory opsins is reduced or eliminated. In one embodiment, the modified organism comprises an animal wherein expression of one or more native chemosensory opsins is reduced, for example by gene knockout or gene knockdown techniques known in the art. Exemplary techniques include, for example, by CRISPR/Cas9 genome editing, short interfering RNA (siRNA), short hairpin (shRNA), and other methods known in the art In various embodiments, the modified organism of the invention comprises a knockout or knockdown of Opn1SW and/or Opn3, or knockout or knockdown of the corresponding ortholog or paralog thereof. In one embodiment, modified organism of the invention comprises a mouse, rat, dog, cat, pig, horse, or other animal comprising a knockout or knockdown of the native Opn1SW, and/or Opn3 or ortholog thereof present therein.

In other embodiments, the modified animal comprises an animal wherein expression of one or more native chemosensory opsins, e.g. OPN1SW or OPN3, is increased in chemosensory cells, i.e. overexpressed, relative to its expression in wild type animals. In one embodiment, a chemosensory opsin, e.g. OPN1SW or OPN3, is expressed ectopically in tissues where it is not normally expressed. Such modifications may increase sensitivity to chemosensory ligands of the modulated opsin.

In one embodiment, the modified organism comprises an animal wherein expression of one or more heterologous chemosensory opsin genes is induced, for example, wherein a chemosensory opsin from another species is expressed in the animal. In one embodiment, the animal is engineered to express OPN1SW and/or OPN3, or an animal homolog or ortholog thereof.

Certain opsin activation assays of the invention utilize cells and/or sensory tissues isolated or derived from animals having the foregoing modulated expression of chemosensory opsins.

In another aspect, the scope of the invention encompasses the use of animals having modulated expression of chemosensory opsins in behavioral studies. For example, by comparing the performance of animals with modulated expression of chemosensory opsins to the performance of wild type animals, insights into the chemosensory properties of materials and the role of chemosensory opsins in chemoperception may be gained. In the context of taste, exemplary behavioral studies using animals, e.g. mice, engineered to have modulated. expression of chemosensory opsins, include, for example, two-bottle or other taste preference tests, lickometer tests, and conditioned taste aversion tests. In the context of olfaction, such animals may be used in tests such as the habituation/dishabituation test, buried food test, and odor discrimination tests.

In another implementation, the scope of the invention encompasses the use of animals comprising modified expression of chemosensory opsins in electrophysiological assays, wherein responses to chemosensory molecules, e.g. tastants and olfactants, are measured, for example, for determining how modulation of chemosensory opsins impacts taste or smell compared to wild type animals.

### IV. EXAMPLES

### Example 1. Opsin Activation by Known Tastants

Human transcriptomics data was mined to identify putative taste receptor proteins. Two opsins, OPN1SW, and OPN3 were found to be expressed in taste buds. However, the role of these opsins in taste perception has not been previously explored. TANGO detection elements employing each of these opsins, in both human and mouse isoforms, were created in HTLA cells (a HEK293 cell line stably expressing a tTA-dependent luciferase reporter and a β-arrestin2-TEV fusion gene), based on protocols in Kroeze et al., 2015. PRESTO-Tango as an open-source resource for interrogation of the druggable human GPCRome. Nat Struct Mol Biol 22, 362-369.

In the TANGO assay, the mouse opsin coding sequence with an N-terminal FLAG tag was synthesized. The construct was cloned into a TANGO vector (Addgene, Waterton MA) at the ClaI site (FLAG::opsin). The HTLA cells were transfected in a 6-well format with LIPOFECTAMINE 2000™ (Thermo Fisher Scientific, Inc, Waltham MA, USA) according to the manufacturer's instructions. 6-8 hours post transfection cells weret split 1:3 and the media was switched to DMEM+ 2.5% fetal bovine serum (Thermo Fisher Scientific Inc., Waltham MA, USA). Cells were allowed to grow for 48 hours before splitting the cells onto poly-L-lysine-coated 96-well clear, flat bottom white polystyrene plates in serum-free media. The next day, chemical solutions diluted in TANGO assay buffer (20 mM HEPES, 1× HBSS, pH 7.40) were added to the cells. After 4 hours, the medium and drug solutions were removed and replaced with 40 mL per well of BRIGHTGLO™ reagent (Promega Corporation, Madison WI) diluted 20-fold with TANGO assay buffer. Plates were incubated for 20 minutes at room temperature in the dark before measuring luminescence with a plate reader (Molecular Devices, iD3). The normalized response was calculated using following formula: RLU (relative luminescence units)=test compound RLU−average vehicle control RLU.

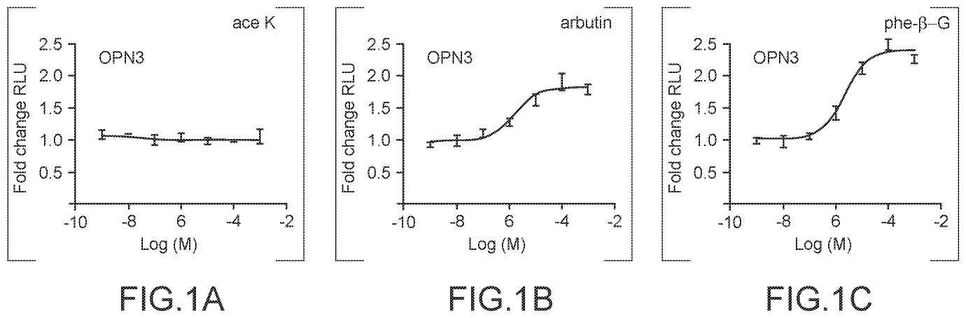

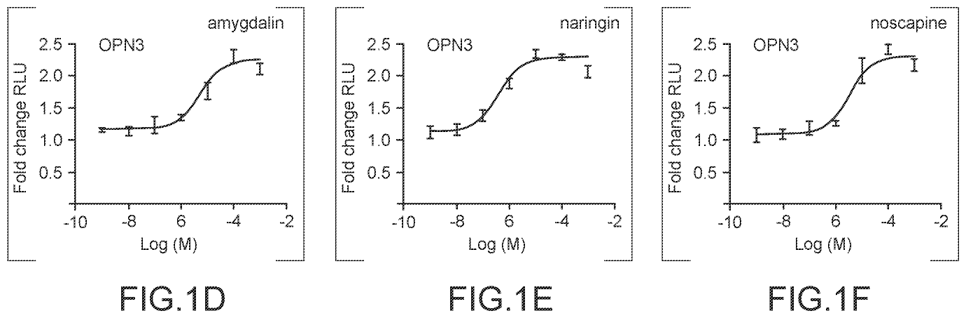

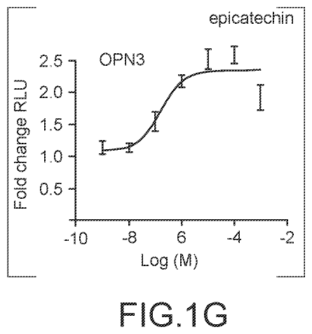

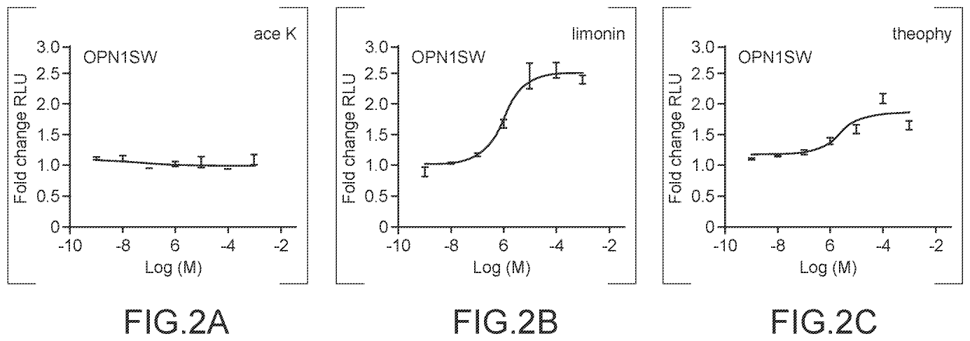

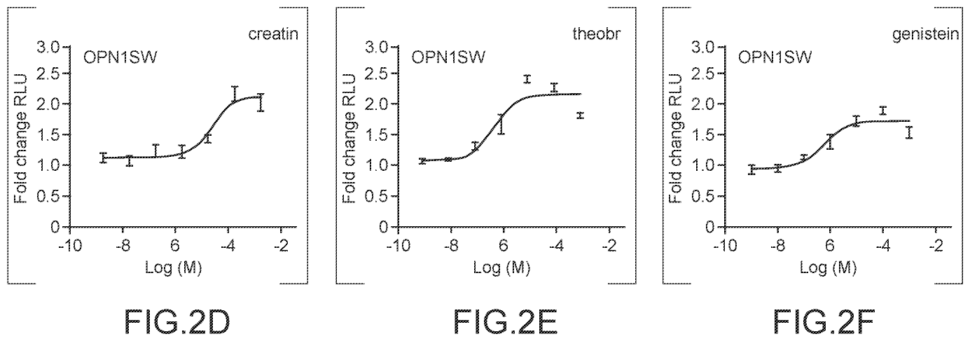

The TANGO detection elements were assayed with a range of tastants with known flavor characteristics. Activation of OPN3 was achieved by several tastants, as shown in FIG. 1A-1G. Activation of OPN1SW was achieved by several tastants, as shown in FIG. 2A-2F.

### Example 2. Opsin Activation by Tastants Confirmation Assays

These foregoing TANTO results were further confirmed using a second, a calcium flux GPCR activation assay employing Fluo-8 dye, as follows. For this intracellular Ca²⁺ measurement, opsin-expressing HEK293T cells were seeded onto 96-well plates (4×104 cells per well) in 100 μl of culture medium with reduced serum contents (0.5% of FBS) per well and incubated overnight under standard culture conditions. [Ca2+]i level was assessed the next day after stimulation with bitter compounds used at a different concentration with the SCREEN QUEST Fluo-8 No Wash Calcium Assay Kit (AAT Bioquest, Inc., Sunnyvale, CA, USA) according to the supplier's protocol. Calcium flux was monitored simultaneously by the change in fluorescence (Ex/Em=490/520 nm) following stimulation and with respect to the background fluorescence. [Ca^{2+]}i was monitored for a period of three minutes, starting before the addition of compounds to the wells and continuing during measurement of the fluorescent signal following cells' stimulation. The obtained fluorescence reads were normalized to the results obtained after stimulation of the cells with the compound solvent.

The calcium dye OPN3 detection elements were significantly activated in a concentration dependent manner by arbutin, amygdalin, phe-β-Glu, naringin, noscapine, and epicatechin, similarly to TANGO results of FIG. 1A-1G (data not shown). The calcium dye OPN1SW detection elements were significantly activated in a concentration dependent manner by theobromine, theophylline, creatinine, limonin, and genistein, similarly to TANGO results of FIG. 2A-2F (data not shown).

### Example 3. Mouse Behavioral Studies

Knockout mice were obtained for OPN1SW and OPN3. Two independent OPN3 knockout mice lines were tested for reaction to naringin by a mouse taste preference assay. A Lickometer device (MS-160, Di Log Instruments, Inc.,Tallahassee, FL, USA) was used to train mice and then measure their licking responses to different tastants in brief-access trials. On a given trial, each sipper tube was filled with a solution and made available to a mouse via a small port that opened to a metal/plexiglass chamber housing the animal. The mouse chamber of the lickometer was modified for these studies by adding a plexiglass barrier that restricted the free movement of animals to an approximate 10 by 15 cm area facing the fluid access port. A computer running software that communicated with the lickometer recorded tongue contacts with the metal tip of the sipper tube during licking and the time intervals (precision=1 ms) between consecutive licks (inter-lick intervals). The amount of time that the sipper tube was available through the access port could be programmed and was controlled by the opening of a normally closed, computer-actuated shutter. This feature facilitated presentation of solutions to a mouse for only a few seconds to support measurements of brief-access licking behavior. By indexing initial licking responses to stimuli over short periods, brief-access procedures intend to focus on oral sensory influences and avoid any post-ingestive effect. Groups of three to four mice were run in these studies at one time. Groups always included equal numbers of mutant and wild-type mice to control for temporal factors.

Training. Mice were restricted from drinking water for 20-22 hours prior to the onset of training. During training, experimentally naive mice learned to receive fluid in the lickometer over a 3-day period. On the first and second day of training, mice were given 30 minutes free access to a single sipper tube of water to familiarize them with accessing fluid in the lickometer device. Each 30 minute period began when the mouse made its first lick on the sipper tube; mice were allowed 30 minutes to make their first lick. On the third day of training, mice were presented with 10 second presentations (i.e., trials) of water to familiarize them with receiving and consuming fluid under a brief-access procedure in the lickometer. On each trial, the shutter that blocked access to the sipper tube was opened and mice were given 10 seconds to lick, and lick data were recorded by the lickometer computer. A response of zero licks was recorded and the trial terminated if a mouse failed to make any lick within 10 seconds of shutter opening. At the end of the trial, the shutter gently closed to block access to the sipper tube and the lickometer advanced to the next trial in the tube presentation sequence with an inter-trial interval of 5 seconds. The intent on training days was to have mice consume all of their daily water in the lickometer devices. However, additional free access to water, for approximately 1 hour via overhead bottle, was given to individual mice following daily training.

Testing. Mice were restricted from drinking water for at least 22 hours prior to the onset of testing. For studies, individual Opn3KO, Opn1swKO, Opn4KO, Opn5KO and wild-type mice were evaluated for brief-access licking responses with different agonists. Mice were randomly presented sipper tubes that delivered four different concentrations of tastant solutions or suspensions, as well as the vehicle. Each sipper tube was presented one at a time for a 5-second sampling trial. There was no ‘rinse’ in between trials. As with training sessions, intertrial intervals were of 5 seconds and test sessions were of 10 minutes in duration. The order of presentation of the sipper tubes was randomized and each concentration was presented 5 times per session. The experiments were repeated on two different weeks intercepted by a one-week washout period. As brief access assay limit the amount of solution ingested by the mice, no evidence of post-ingestive effects of consumed bitter chemicals was indicated when lick rate during test sessions was compared with lick rates obtained on water-training sessions.

Data analysis. For the mouse brief-access taste assay, the number of licks per trial for each concentration of bitter solution/suspension or vehicle was collected and averaged across animals in each experimental group (n=6). For comparisons between genotypes, data were normalized by dividing the number of licks emitted by a mouse for each trial by licks for the vehicle control trials, to yield a ‘lick ratio’. The “lick numbers” obtained for deionised water and four concentrations of tastant solution were recorded. “Lick ratios” (% inhibition of licking compared to deionised water) were calculated by dividing the number of licks of the test solution (Test solution) by the average number of licks for deionised water. No data recorded was discarded. Curve fitting (Prism; GraphPad Software, San Diego, California, USA) was performed on the data: Lick ratio=Number of licks to each test solution/Mean number of licks to water×100%.

As some of the compounds tested required using organic solvent vehicles, it was tested whether the vehicles themselves were orally aversive to mice. Ethanol and DMSO were tested over a range of concentrations. Ethanol and DMSO up to concentrations of 1% (v/v) had no measurable effect on lick rate compared with water alone.

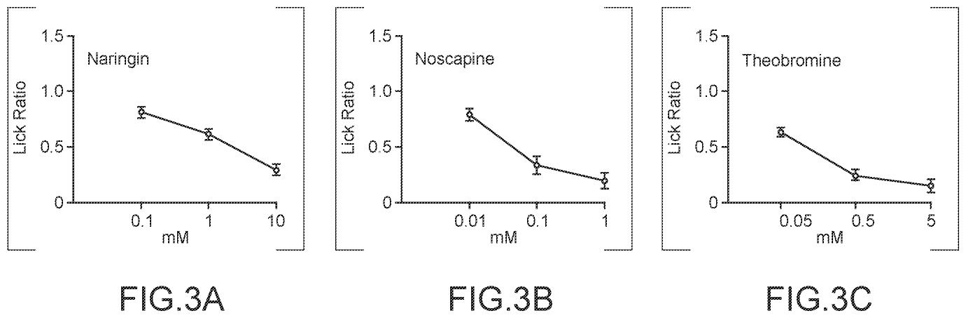

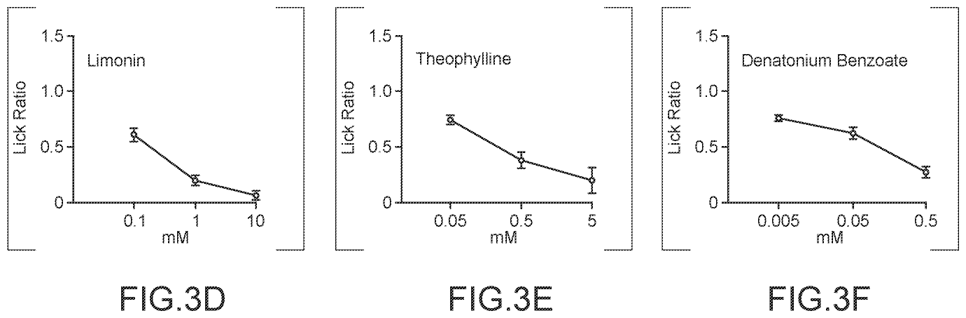

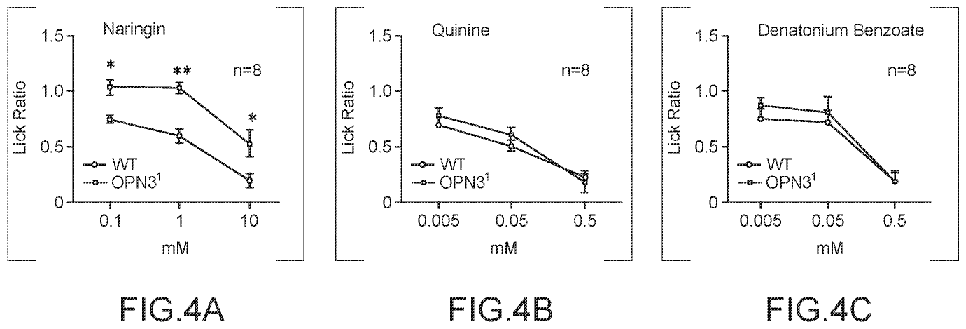

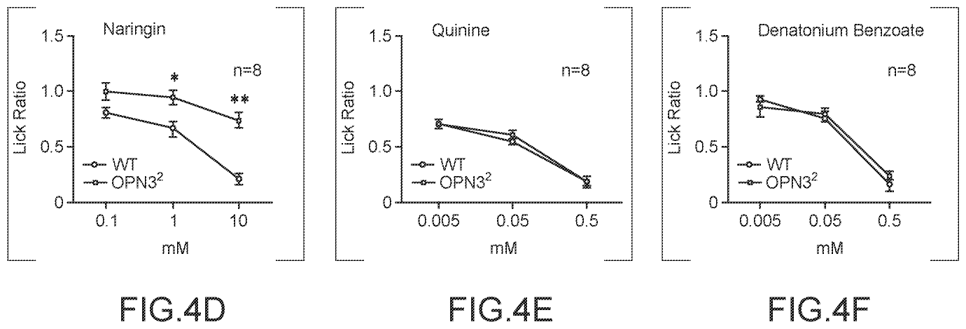

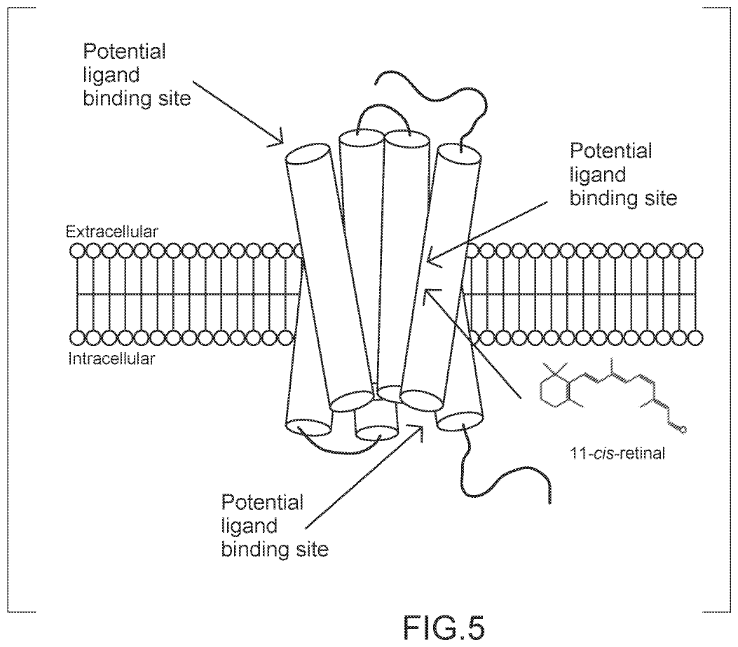

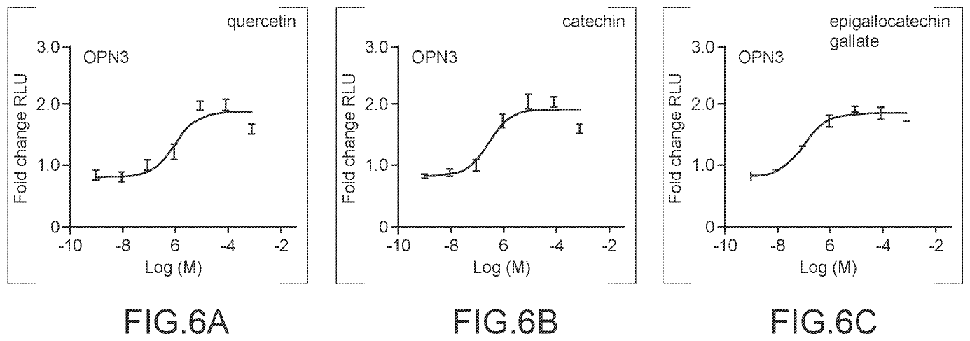

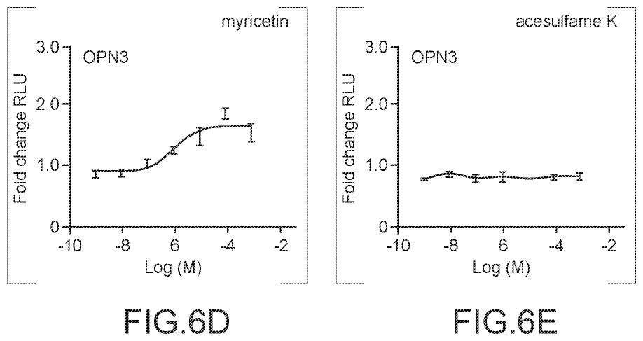

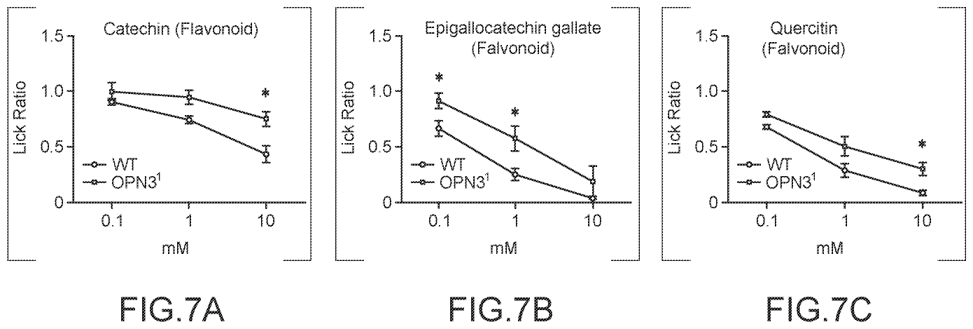

Results. In wild type mice, aversion to bitter tastants increased with increasing tastant concentrations (FIG. 3A-3F). Compared to wild type mice, OPN3 knockout mice had a reduced aversion to the bitter flavor of Naringin (FIG. 4A), demonstrating that OPN3 is utilized in taste perception in mammals. In addition, the gustatory responses of OPN3 knockout mice to additional flavonoids was tested (catechin, Epigallocatechin, and quercetin), which activated OPN3 using the in vitro TANGO assay (FIG. 6A-6E). In taste preference tests, it was found that OPN3 knockout mice had a reduced aversion to the bitter flavor of these structurally related flavonoids (FIG. 7A-7C).

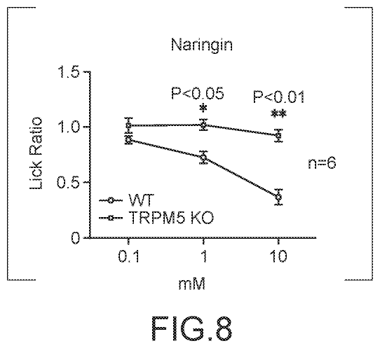

The reaction of TRPM5 knockout mice (taste blind mice) for Naringin was also tested, since TRPM5 is required for sweet, bitter and umami taste perception. It was found that TRPM5 knockout mice lost the aversive response to the bitter flavor of naringin (FIG. 8), indicating that sensing of flavonoids by OPN3 is TRPM5 dependent.

### Example 4. Mouse Anatomical Studies

Explants of mouse tongue comprising taste buds were assayed by in situ hybridizations using an RNAscope probe for OPN3. Fungiform papillae and foliate papillae from the mouse tongue were stained for OPN3 probe expression and co-stained with a probe for TRPM5, which is a cation channel required for bitter, sweet and umami taste in mice.

To detect expression of opsin mRNAs in the mice taste organs by reverse transcription PCR (RT-PCR), total RNA was estracted from each of the tongue sample, using RNEASY™ mini kit (Qiagen, GmbH, Hilden, Germany). DNase I treatment was applied to eliminate traces of DNA during the procedure. cDNA was prepared using SUPERSCRIPT™ III Reverse Transcriptase (Thermo Fisher Scientific, Walham MA, USA) using OLIGODOT™ primers. For RT-PCR, amplified was performed for 40 cycles using PHUSION™ DNA polymerase (New England BioLabs, Ipswitch MA, USA),

These hybridization studies showed that OPN3 co-localizes with TRPM5 in type II taste receptor cells.

### Example 5. Expression of Opsins in the Tongue and Olfactory Epithelium

RNA was isolated from wild-type mouse tongues and olfactory epithelia. Equal concentrations of RNA were used to prepare cDNAs, and RT-PCR was performed using Opn3, Opn1SW, and Opn4 primers spanning exon-exon junction. β-actin and eif2 (eukaryotic elongation factor-2) were used as loading controls. It was observed that Opn3, Opn1SW and Opn4 transcripts were present in both the tongue and olfactory epithelium tissue.

In sum, the behavioral and anatomical animal data demonstrate that opsins represent a previously unappreciated class of taste receptors active in mammals.

All patents, patent applications, and publications cited in this specification are herein incorporated by reference to the same extent as if each independent patent application, or publication was specifically and individually indicated to be incorporated by reference. The disclosed embodiments are presented for purposes of illustration and not limitation. While the invention has been described with reference to the described embodiments thereof, it will be appreciated by those of skill in the art that modifications can be made to the structure and elements of the invention without departing from the spirit and scope of the invention as a whole.

What is claimed is:

1. A chemosensory opsin activation detection element, comprising
one or more chemosensory opsins;
wherein the detection element is configured to generate a measurable signal in response to activation by binding of a chemical ligand.

2. The chemosensory opsin activation detection element of claim 1, wherein
the one or more chemosensory opsins comprises human OPN1SW, a variant thereof, or a homolog, paralog, or ortholog thereof.

3. The chemosensory opsin activation detection element of claim 1, wherein
the one or more chemosensory opsins comprises human OPN3, a variant thereof, or a homolog, paralog, or ortholog thereof.

4. The chemosensory opsin activation detection element of claim 1, wherein
the chemosensory opsin activation detection element comprises a cell.

5. The chemosensory opsin activation detection element of claim 4, wherein
the cell comprises a cell derived from chemosensory tissue of an animal.

6. The chemosensory opsin activation detection element of claim 4, wherein
the cell comprises a cell engineered to express the one or more chemosensory opsins.

7. The chemosensory opsin activation detection element of claim 1, wherein
the chemosensory opsin activation detection element is loaded with or expresses a calcium indicator.

8. The chemosensory opsin activation detection element of claim 1, wherein
the chemosensory opsin activation detection element is loaded with or expresses a cAMP indicator.

9. The chemosensory opsin activation detection element of claim 1, wherein
the chemosensory opsin activation detection element is loaded with or expresses an indicator of electrical activity.

10. The chemosensory opsin activation detection element of claim 1, wherein
the chemosensory opsin activation detection element is configured to generate the detectable signal by beta-arrestin recruitment to the activated chemosensory opsin.

11. An assay system, comprising
a container element wherein a plurality of chemosensory opsin activation detection elements of any of claims 1-10 are disposed; and
a detection device.

12. The assay system of claim 11, wherein
the container element is a well of a multiwell plate.

13. The assay system of claim 11, wherein
the detection device is an optical detector.

14. The assay system of claim 11, wherein
the detection device is a detector of electrical activity.

15. A method of measuring the activation of a chemosensory opsin by the binding of a chemical ligand, comprising
providing a sample to one or more chemosensory opsin activation detection elements, wherein the chemosensory opsin activation detection elements comprise a cell or other composition of matter comprising a selected chemosensory opsin, configured such that activation of the chemosensory opsin by a chemical ligand generates a detectable signal;
wherein the sample comprises or putatively comprises an activating ligand of the chemosensory opsin;
wherein sample is provided to the chemosensory opsin activation detection elements under conditions suitable for ligand binding to, or interaction with, the chemosensory opsin proteins present therein; and
measuring signal generated by the one more chemosensory opsin activation detection elements.

16. The method of claim 15, wherein
the one or more chemosensory opsins comprises human OPN1SW, a variant thereof, or a homolog, paralog, or ortholog thereof.

17. The method of claim 15, wherein
the one or more chemosensory opsins comprises human OPN3, a variant thereof, or a homolog, paralog, or ortholog thereof.

18. The method of claim 15, wherein
the chemosensory opsin activation detection element comprises a cell.

18. The method of claim 18, wherein
the cell comprises a cell derived from chemosensory tissue of an animal.

19. The method of claim 18, wherein
the cell comprises a cell engineered to express the one or more chemosensory opsins.

20. The method of claim 15, wherein
the detectable signal generated in response to chemosensory opsin activation is an optical signal.

21. The method of claim 15, wherein
the detectable signal generated in response to chemosensory opsin activation is an electrical signal.

22. The method of claim 15, wherein
the detectable signal generated in response to chemosensory opsin activation is detected by any of calcium flux, cAMP concentration, electrical activity, neurotransmitter release, or an optical signal generated by beta-arrestin recruitment to the activated chemosensory opsin.

23. The method of claim 15, wherein
the sample comprises a tastant or putative tastant.

24. The method of claim 15, wherein
the sample comprises an olfactant or putative olfactant.

25. The method of claim 15, wherein
the sample comprises a putative ligand of the chemosensory opsin; wherein generation of a detectable signal indicates that the putative ligand is an activator of the chemosensory opsin.

26. The method of claim 15, wherein
the sample comprises a modified form of a known ligand of the chemosensory opsin.

27. The method of claim 15, wherein
the sample comprises a known ligand of the chemosensory opsin and a putative chemosensory modulator.

28. The method of claim 27, wherein
the putative chemosensory modulator comprises a composition that masks or inhibits bitter flavor.

29. An engineered animal, wherein
expression of one or more chemosensory opsins in chemosensory tissues of the animal is modulated relative to wild type animals.

30. The engineered animal of claim 29, wherein
the chemosensory opsin is OPN1SW.

31. The engineered animal of claim 29, wherein
the chemosensory opsin is OPN3.

32. The engineered animal of claim 29, wherein
the animal is engineered to overexpress the one or more chemosensory opsins.

33. The engineered animal of claim 29, wherein
the animal is engineered to overexpress a heterologous chemosensory opsin.

34. The engineered animal of claim 29, wherein
the animal is engineered such that expression of one or more native chemosensory opsins in chemosensory tissue is reduced or eliminated.

35. The engineered animal of claim 29, wherein
the animal is a mouse.

36. A cell, wherein
the cell is derived from the animal of any of claims 29-35.

37. The cell of claim 36, wherein
the cell comprises any of explanted chemosensory tissue, cultured primary chemosensory cells, cultured cells, a spheroid or organoid, or other cell culture product.
